(19) United States
(12) Patent Application Publication
(10) Pub. No.: US 2026/0060538 A1
(43) Pub. Date: Mar. 5, 2026

(54) SYSTEM AND METHOD OF PERFORMING DIGITAL IMAGING USING NONVISIBLE LIGHT

(71) Applicant: Alcon Inc., Fribourg (CH)

(72) Inventors: Michael Sam Cardamone, Keller, TX (US); Sumit Paliwal, Irvine, CA (US); Lingfeng Yu, Rancho Santa Margari, CA (US); John Park, Irvine, CA (US)

(21) Appl. No.: 19/316,066

(22) Filed: Sep. 2, 2025

Related U.S. Application Data
(60) Provisional application No. 63/690,935, filed on Sep. 5, 2024.

(51) Int. Cl. A61B 3/10; A61B 3/12; G06T 5/50; H04N 23/11; H04N 23/13; H04N 23/74

(52) U.S. Cl. CPC A61B 3/102; A61B 3/12; G06T 5/50; H04N 23/11; H04N 23/13; H04N 23/74; G06T 2207/10048; G06T 2207/20212; G06T 2207/30041

(57) ABSTRACT

A visualization system for use during an ophthalmic procedure. The system includes at least one illumination source configured to emit in a visible and an invisible light spectrum, a light filter configured to allow light within a filtered light spectrum, and an image sensor assembly configured to detect first and second sets of images from the visible and invisible light spectrums, respectively. The system also includes an electronic control unit (ECU) in communication with the at least one illumination source and the image sensor assembly. The ECU is programmed to activate the at least one illumination source to emit light in the visible and the invisible light spectrum and receive image data from the image sensor assembly representative of the first and second sets of images. The controller is also programmed to generate and display at least one composite image based on the first and second sets of images.

### INTRODUCTION

The present disclosure relates to systems and methods for generating images of an eye based on a combination of images captured in a visible light spectrum and images captured from an invisible light spectrum.

Multiple different types of imaging technologies can capture images of the eye, such as optical coherence tomography (“OCT”). OCT provides a noninvasive imaging technology using low-coherence interferometry to generate high-resolution images of an ocular structure. OCT imaging functions partly by measuring the echo time delay and magnitude of backscattered light. Images generated by OCT are useful for many purposes, such as identification and assessment of ocular diseases.

### SUMMARY

Disclosed herein is a visualization system for use during an ophthalmic procedure on a target eye. The system includes at least one illumination source configured to emit light in a visible light spectrum and in an invisible light spectrum, a light filter located along a reflected light path and configured to allow for the transmission of light within a filtered light spectrum, and an image sensor assembly positioned along the reflected light path and configured to detect a first set of images from the visible light spectrum and a second set of images from the invisible light spectrum. The system also includes an electronic control unit (ECU) in communication with the at least one illumination source and the image sensor assembly. The ECU is programmed to activate the at least one illumination source to emit light in the visible light spectrum and in the invisible light spectrum and receive image data from the image sensor assembly representative of the first set of images and the second set of images. The controller is also programmed to generate at least one composite image based on the first set of images and the second set of images and display the at least one composite image.

In one aspect of the disclosure the invisible light spectrum includes an infrared light spectrum.

In one aspect of the disclosure the invisible light spectrum includes an ultraviolet light spectrum.

In one aspect of the disclosure the image sensor assembly includes a first sensor configured to capture a first perspective of the target eye and a second sensor configured to capture a second perspective of the target eye and the first set of images includes a first set of first perspective images and a first set of second perspective images and the second set of images includes a second set of first perspective images and a second set of second perspective images.

In one aspect of the disclosure the ECU is programmed to generate the at least one composite image by organizing the first set of first perspective images and the second set of first perspective images in an alternating sequential pattern and organizing the first set of second perspective images and the second set of second perspective images in an alternating sequential pattern.

In one aspect of the disclosure the ECU is programmed to generate the at least one composite image by organizing the first set of images in an alternating sequential pattern with the second set of images.

In one aspect of the disclosure the ECU is programmed to generate the at least one composite image by organizing corresponding images from the first set of images adjacent corresponding images from the second set of images.

In one aspect of the disclosure the ECU is programmed to generate the at least one composite image by organizing corresponding image pairs from the first set of images and the second set of images in an overlaid image configuration.

In one aspect of the disclosure the ECU is programmed to generate the at least one composite image by organizing corresponding image pairs from the first set of images and the second set of images into a blended image configuration.

In one aspect of the disclosure the at least one illumination source includes a first light source operable for directing visible light toward the target eye, the first light source including an array of red, green, and blue (RGB) laser diodes and a second light source operable for directing invisible light toward the target eye, the second light source including at least one an IR laser diode or a UV diode.

Disclosed herein is a non-transitory computer-readable storage medium embodying programmed instructions which, when executed by a processor, are operable for performing a method. The method includes illuminating a target eye by directing a visible spectrum light and an invisible spectrum light toward the target eye with at least one illumination source and capturing a first set of images from the visible light spectrum and a second set of images from the invisible light spectrum with an image sensor assembly. The method also includes generating at least one composite image based on the first set of images and the second set of images and displaying the at least one composite image.

In one aspect of the disclosure the image sensor assembly includes a first sensor configured to capture a first perspective of the target eye and a second sensor configured to capture a second perspective of the target eye and the first set of images includes a first set of first perspective images and a first set of second perspective images and the second set of images includes a second set of first perspective images and a second set of second perspective images. Also, generating the at least one composite image includes organizing the first set of first perspective images and the second set of first perspective images in an alternating sequential pattern and organizing the first set of second perspective images and the second set of second perspective images in an alternating sequential pattern.

In one aspect of the disclosure the generating the at least one composite image includes organizing the first set of images in an alternating sequential pattern with the second set of images.

In one aspect of the disclosure the generating the at least one composite image includes organizing corresponding images from the first set of images adjacent corresponding images from the second set of images on a display.

In one aspect of the disclosure the generating the at least one composite image includes organizing corresponding image pairs from the first set of images and the second set of images in one of an overlaid image configuration or a blended image configuration.

Disclosed herein is a method for using a visualization system during an ophthalmic procedure. The method includes illuminating a target eye by directing a visible spectrum light and an invisible spectrum light toward the target eye with at least one illumination source and capturing a first set of images from the visible light spectrum and a second set of images from the invisible light spectrum with an image sensor assembly. The method also includes generating at least one composite image based on the first set of images and the second set of images and displaying the at least one composite image.

In one aspect of the disclosure the image sensor assembly includes a first sensor configured to capture a first perspective of the target eye and a second sensor configured to capture a second perspective of the target eye and the first set of images includes a first set of first perspective images and a first set of second perspective images and the second set of images includes a second set of first perspective images and a second set of second perspective images. Also, generating the at least one composite image includes organizing the first set of first perspective images and the second set of first perspective images in an alternating sequential pattern and organizing the first set of second perspective images and the second set of second perspective images in an alternating sequential pattern.

In one aspect of the disclosure generating the at least one composite image includes organizing the first set of images in an alternating sequential pattern with the second set of images.

In one aspect of the disclosure generating the at least one composite image includes organizing corresponding images from the first set of images adjacent corresponding images from the second set of images on a display.

In one aspect of the disclosure generating the at least one composite image includes organizing corresponding image pairs from the first set of images and the second set of images in one of an overlaid image configuration or a blended image configuration.

### BRIEF DESCRIPTION OF THE DRAWINGS

FIG. 1 is a schematic illustration of an exemplary surgical suite configured with a visualization system as set forth in detail herein.

FIG. 2A is a front view illustration of a target eye that can be visualized within the surgical suite shown in FIG. 1.

FIG. 2B is a cross-sectional side view illustration of the target eye depicted in FIG. 2B.

FIG. 3 is a flowchart describing an embodiment of a method for generating a composite image of the target eye of FIGS. 2A and 2B using the visualization system of FIG. 1.

The foregoing and other features of the present disclosure are more fully apparent from the following description and appended claims, taken in conjunction with the accompanying drawings.

### DETAILED DESCRIPTION

Embodiments of the present disclosure are described herein. It is to be understood, however, that the disclosed embodiments are merely examples and other embodiments can take various and alternative forms. The figures are not necessarily scaled. Some features could be exaggerated or minimized to show details of particular components. Therefore, specific structural and functional details disclosed herein are not to be interpreted as limiting, but merely as a representative basis for teaching one skilled in the art to variously employ the present disclosure. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

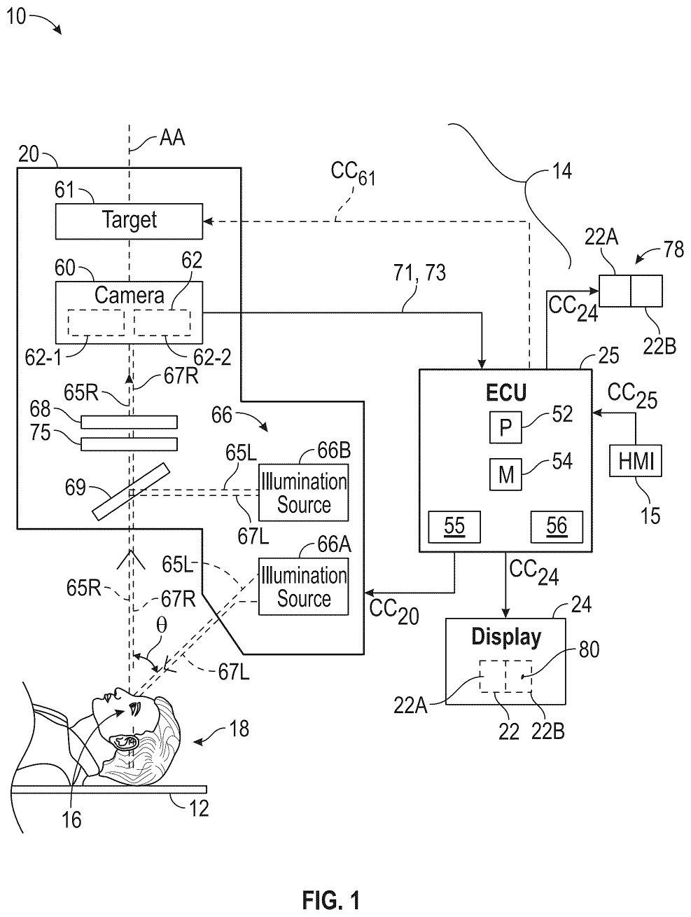

Referring to the drawings, wherein like reference numbers refer to like components, a representative surgical suite 10 is depicted schematically in FIG. 1. The surgical suite 10 may be equipped with a multi-axis surgical robot (not shown), an operating platform 12 such as an adjustable table or chair, and a visualization system 14 configured as set forth herein. The surgical suite 10 can be used when performing a surgical or diagnostic procedure on a target eye 16 of a patient 18. The target eye 16, being the particular subject surgical site in accordance with the following disclosure, is therefore referred to hereinafter as a target eye 16 for clarity.

As contemplated herein, representative ophthalmic procedures performable in the surgical suite 10 of FIG. 1 include lens replacement surgeries, e.g., cataract surgeries or refractive lens exchanges (RELs), diagnoses or treatments of conditions of the target eye 16 such as capsular tears, or the visualization of the internal limiting membrane (ILM) (not shown) of the target eye 16 or other ocular anatomy. During such procedures, a surgeon may have difficulty visualizing implantable devices and/or the ocular anatomy. While lens replacement surgeries are described in the examples that appear below, those skilled in the art will appreciate that other ophthalmic surgeries or in-office procedures may similarly benefit from the present teachings.

The visualization system 14 shown in FIG. 1 in one or more embodiments may be connected to or in communication with an ophthalmic microscope 20 through which the surgeon is able to view the target eye 16. Alternatively, the visualization system 14 may be partially or fully integrated with the hardware and software of the ophthalmic microscope 20. Using the visualization system 14, the surgeon is able to view one or more composite images 22 of the target eye 16, which may be viewed within the surgical suite 10 via a corresponding high-resolution medical display 24, and possibly through ocular pieces (not shown) of the ophthalmic microscope 20.

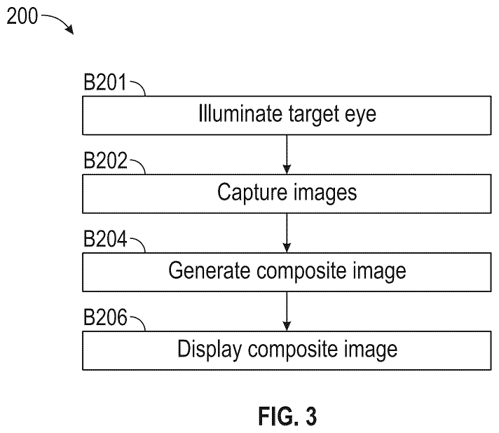

An electronic control unit (ECU) 25 is also present within the exemplary surgical suite 10 of FIG. 1. The ECU 25, which within the scope of the disclosure is used with or as an integral part of the visualization system 14, is programmed in software and equipped in hardware, i.e., configured, to execute computer readable instructions embodying a method 200, a representative implementation of which is described below with reference to FIG. 3. Execution of the method 200 in turn allows the surgeon to better visualize certain features of the target eye 16 when diagnosing or treating the target eye 16, as noted above.

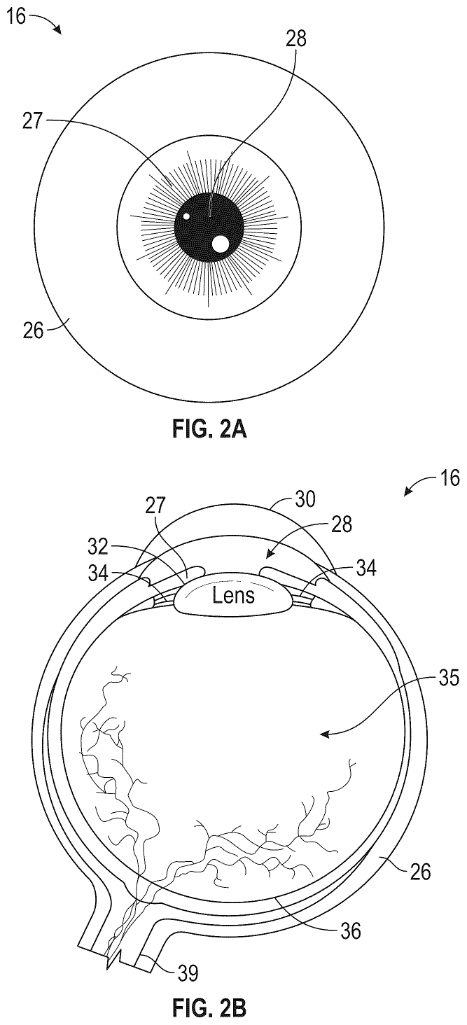

Referring briefly to FIGS. 2A and 2B, the target eye 16 includes an iris 27 that is surrounded by sclera 26. A pupil 28 is centrally located within/surrounded by the iris 27. As shown in FIG. 2B, the target eye 16 also includes a cornea 30 spanning and protecting the iris 27 and the pupil 28. Light admitted through the pupil 28 passes through a natural lens 32, which in turn is connected to the surrounding anatomy of the target eye 16 via ciliary muscles 34. Also shown in FIG. 2B is the vitreous cavity 35, which is filled with vitreous humor (not shown), a retina 36 lining posterior portions of the vitreous cavity 35, and the optic nerve 39 disposed at the rear of the vitreous cavity 35 opposite the lens 32.

Although the ECU 25 shown in FIG. 1 is depicted as a unitary box for illustrative clarity and simplicity, the ECU 25 within the scope of the disclosure could include one or more networked devices each with a central processing unit or other processor (P) 52 and sufficient amounts of memory (M) 54, including a non-transitory (e.g., tangible) storage medium that participates in providing data/instructions that may be read by the processor(s) 52. Instructions embodying image combination 55 may be stored in the memory 54 and executed by the processor 52 to perform the various functions described herein, thus enabling the present method 200 exemplified in FIG. 3.

The memory 54 may take many forms, including but not limited to non-volatile media and volatile media. Non-volatile media may include optical and/or magnetic disks or other persistent memory, while volatile media may include dynamic random-access memory (DRAM), static RAM (SRAM), etc., any or all which may constitute a main memory of the ECU 25. Input/output (I/O) circuitry 56 may be used to facilitate connection to and communication with the various peripheral devices used during the ophthalmic procedure, inclusive of the various hardware of the visualization system 14 of FIG. 1.

Other hardware not depicted but commonly used in the art may be included as part of the ECU 25, including but not limited to a local oscillator or high-speed clock, signal buffers, filters, etc. A human machine interface (HMI) 15 may be included within the structure of the visualization system 14 to allow the surgeon to interact with the ECU 25, e.g., via input signals (arrow CC₂₅). The ECU 25 may also control the ophthalmic microscope 20 directly, e.g., via microscope control signals (arrow CC₂₀), or via the input signals (arrow CC₂₅) in different embodiments. Various implementations of the HMI 15 may be used within the scope of the present disclosure, including but not limited to a footswitch, a touch screen, buttons, control knobs, a speaker for voice activation, etc. The ECU 25 of FIG. 1 may be configured to communicate via a network (not shown), for instance a serial bus, a local area network, a controller area network, a controller area network with flexible data rate, or via Ethernet, Wi-Fi, Bluetooth™, near-field communication, and/or other forms of wired or wireless data connection.

Still referring to FIG. 1, the visualization system 14 contemplated herein includes a camera assembly 60, have an image sensor 62. The camera assembly 60 is configured to detect light in a specific portion of the electromagnetic spectrum. In one example, the camera assembly 60 is configured or “tuned” to detect incident reflected light 65R in the human-visible spectrum, which is typically defined as corresponding to wavelengths of about 380 nanometers (nm) to about 750 nm. The camera assembly 60 is also configured to detect reflected light 67R in the near infrared (“NIR”) range, which is typically defined for the purposes of executing the present strategy as “eye-safe” wavelengths of about 780 nm to about 1.4 micrometers (μm). A filter 68 may be used to restrict only the desired reflected light 65R and 67R to reach the camera assembly 60 with or without a polarizer 75. In one example, the filter 68 is a cyan filter to aid in visualizing through blood in the target eye 16. In a possible construction, the camera assembly 60 may be embodied as complementary metal-oxide-semiconductor (CMOS) image sensors, e.g., commercially available CMOS imagers from Teledyne Technologies of Thousand Oaks, CA.

The visualization system 14 illustrated in FIG. 1 also includes a first illumination source 66A and a second illumination source 66B that are each capable of generating a wideband spectrum of illumination that includes the visible light spectrum and the invisible light spectrum. The illumination sources 66A and 66B are configured to emit light toward the target eye 16 in a designated portion of the electromagnetic spectrum with the first illumination source 66A providing off-axis illumination and the second illumination source 66B providing co-axial illumination by utilizing a beam splitter 69. Specifically, a visible spectrum light 65L, i.e., human-visible light and an invisible light spectrum 67L, such as NIR and or ultraviolet (“UV”). The first and second illumination sources may also each be capable of producing polarized light.

Various approaches may be used to implement the wideband illumination source 66. For instance, the visible spectrum light 65L from the wideband illumination source 66 may be generated with a red (R) laser diode, a green (G) laser diode, and a blue (B) laser diode, e.g., as an RGB laser diode array configured to generate the visible spectrum light 65L as white light. Similarly, the invisible spectrum light 67L from the wideband illumination source 66 could be embodied as one or more laser diodes, such as NIR laser diodes or UV laser diodes.

During the illustrated surgical procedure, the visible and invisible spectrum light 65L and 67L reflect off the target eye 16 at an angle θ from the first illumination source 66A or co-axially with the second illumination source 66B. The reflected visible and invisible light 65R and 67R is directed along an optical axis AA extending along an axis of the pupil 28 of FIG. 2A and a suitable optical target (“Target”) 61. The optical target 61 may be static, or the optical target 61 may have one or more parameters, e.g., size, font, appearance, etc., that the ECU 25 may adjust via target control signals (arrow CC₆₁).

The reflected visible and invisible light 65R and 67R are directed toward the camera assembly 60. In one example, the camera assembly 60 includes a sensor 62 that may have a first perspective camera sensor 62-1 and a second perspective camera sensor 62-2 depending on if digital goggles 78 or a display 24 are being used with the surgical suite 10. The camera assembly 60 thereafter output corresponding visible spectrum images 71 and invisible spectrum images 73 to the ECU 25 for further processing.

In executing the above-noted instruction set embodying the method 200 or variations thereof, the ECU 25 of FIG. 1 is rendered operable for assisting in the real-time visualization of the target eye 16 by capturing both the visible spectrum images 71 and invisible spectrum images 73 that are used to generate at least one composite image 22. To this end, the visualization system 14 facilitates imaging tissue structures deeper into the eye, such as the ability to see through the retina to underlaying structures of the eye. The visualization system 14 also facilitates imaging tissue structures in low light scenarios by reducing photo toxicity and allowing for low light illuminations of the target eye 16. Furthermore, the visualization system 14 facilitates imaging of different pathologies, such as thicker tissues to determine the presence of blood vessels or visualization of the retina by seeing through blood if present in the target eye 16.

Referring once again to FIG. 1, the ECU 25 is configured to capture both visible spectrum images 71 and invisible spectrum images 73 of the target eye 16. Thereafter, the ECU 25 combines corresponding visible spectrum images 71 and invisible spectrum images 73 to construct the at least one composite image 22 of the target eye 16. After this occurs, the display 24 is commanded by the ECU 25, e.g., via electronic display control signals (arrow CC₂₄), to display the composite image 22 on the display 24. The ECU 25 utilizes the visible spectrum images 71 and the invisible spectrum images 73 as discussed below to allow an operator of the visualization system 14 to benefit from the unique advantages that each image type provide when generating the composite image 22.

In one example, the ECU 25 can generate the at least one composite image 22 and display it by organizing a visible spectrum image 71 and a corresponding invisible spectrum image 73 adjacent to or next to each other, such as in a side-by-side orientation on the display 24. Alternatively, the visible spectrum image 71 and the invisible spectrum image 73 can be displayed in an overlapping configuration, such as a picture-in-picture orientation, with one of the visible spectrum image 71 or the invisible spectrum image 73 being displayed in a larger format and the other being displayed in a smaller format covering or overlapping a portion of the image in the larger format.

In another example, the ECU 25 can generate the at least one composite image 22 and display it by organizing one of the visible spectrum image 71 or the invisible spectrum image 73 interleaved together such that one of the image types is overlaid in a transparent manner on a corresponding one of the other image types. This approach can highlight features with greater clarity in one of the image types but not the other and vice versa.

In yet another example, the ECU 25 can generate the at least one composite image 22 and display it by organizing the visible spectrum image 71 and the invisible spectrum image 73 in an alternating sequential pattern. With the alternating sequential patten, the at least one composite image 22 includes a single one of the visible spectrum image 71 or the invisible spectrum image 73 displayed at a given time. In one example, the composite image 22 is displayed at rate of 60 frames per second (fps) on the display 24. The alternating sequential pattern of displaying the visible spectrum images 71 relative to the invisible spectrum images 73 results in displaying the visible spectrum images 71 in 30 frames of the 60 fps and displaying the invisible spectrum images 73 in the alternative sequential pattern in the other 30 frames. However, the display rate of the visible and invisible spectrum images 71 and 73 that illustrate the composite image 22 on the display 24 can be greater than or less than 60 fps, such as being greater than or equal to 30 fps or less than or equal to 120 fps. Additionally, while the above example displays the at least one composite image 22 with the visible spectrum images 71 and the invisible spectrum images 73 having an equal number of fps, the fps for forming the composite image 22 could include two or more visible spectrum images 71 for each invisible spectrum image 73 or vice versa.

One feature of generating the at least one composite image 22 and displaying the visible spectrum images 71 and the invisible spectrum images 73 in an alternating sequential pattern, as discussed above, is that an image representative of each of the image types can remain visible to the operator due to presence of vision. The presence of vision resulting from the alternating sequential pattern allows the operator viewing the display 24 or other device to visualize both the visible spectrum image 71 and the invisible spectrum image 73 simultaneously.

In another example, the ECU 25 utilizes digital goggles 78 associated with the ophthalmic microscope 20 to display the at least one composite image 22 in place of or in addition to the display 24. The digital goggles 78 allows for left and right perspective views to be displayed through respective left and right eyepieces on the digital goggles 78. Similar to the alternating sequential pattern described above with the composite image 22 displaying a single image at a given time, the at least one composite image 22 can include separate images created for each of the left and right perspectives views being displayed by the digital goggles 78. As discussed above, the camera assembly 60 can include the first perspective camera sensor 62-1 and a second perspective camera sensor 62-2 for capturing the left perspective image and the right perspective image used to generate the left and right perspective composite images.

In this example, the composite image 22 includes separate images for each both a first perspective composite image 22A and a second perspective composite image 22B with each composite image 22A and 22B displaying an alternating sequential pattern between the visible spectrum images 71 and the invisible spectrum images 73 for each perspective. In one example, the alternating sequential pattern includes displaying the visible spectrum image 71 and the invisible spectrum image 73 in unison for the left and right perspective views on the digital goggles 78. Therefore, only a single image type is displayed at a given time.

In another example, the alternating sequential image pattern on the digital goggles 78 includes displaying the visible spectrum image 71 and the invisible spectrum image 73 in a rotating pattern between the left perspective view and the right perspective view. Therefore, two different image types are being displayed at a given time between the left and right perspective views on the digital goggles 78.

In yet another example, the ECU 25 can generate the at least one composite image 22 and display it by blending together the visible spectrum image 71 and the invisible spectrum image 73. In one example, the at least one composite image 22 is generated by registering or aligning corresponding visible and invisible spectrum images 71 and 73 together. By blending the visible and invisible spectrum images 71 and 73 together to form the composite image 22, points or regions of interest visible in only one of the images 71 or 73 can be highlighted in the composite image 22. Furthermore, artificial intelligence (“AI”) or augmented reality can enlarge locations or regions of interest in the composite image 22.

In addition to creating the at least one composite image 22 from the visible spectrum images 71 and the invisible spectrum images 73, this disclosure is directed to creating digital nondestructive markings 80 of retinal holes/tears or other structures of the target eye 16 utilizing the ECU 25. This approach avoids performing destructive marking, such as by utilizing a laser or endo diathermy. In one example, the nondestructive markings 80 are at least partially based on digital imaging of the target eye 16, through obtaining a corresponding digital image of the target eye 16, such as with an OCT image, illustrating a portion of the retina.

Once the digital image is captured, image registration is performed with at least one of the visible spectrum images 71 or the invisible spectrum images 73. In one example, the digital image, the visible spectrum image 71, and the invisible spectrum image 73 are registered or aligned by identifying features in the images. The identifying features can include scleral blood vessels or retinal blood vessels identified between the different images such that the different image types can be aligned based on the identifying features.

Once the images are registered, the nondestructive marking 80 of a location or region of interest on the retina can appear on at least one of the digital image, the visible spectrum image 71, or the invisible spectrum image 73 as part of the composite image 22. Whether or not the nondestructive marking 80 appears in the visible spectrum image 71 or the invisible spectrum image 73 can depend on a field of view of the image type due to the inherent limitations of each imaging technology. For example, the invisible spectrum images 73 may penetrate deeper into the target eye 16, such as through blood, which the visible spectrum images 71 may not have captured.

Furthermore, during a surgical procedure, the nondestructive marking 80 can be identified on the registration of the digital image, the visible spectrum image 71, or the invisible spectrum image 73 by applying the nondestructive marking utilizing the HMI 15 in communication with the ECU 25 to select a point or region on the registered image corresponding to the point or region of interest. The nondestructive marking 80 can also be generated using a probe or light pipe that utilizes a tip portion as a “target” that can add the digital marking when the “target” is over the spot or region of interest to be marked. The HMI 15 can be used as a trigger to mark the location or the region of interest. The nondestructive marking 80 can be attached to each of the registered images as an overlay in register with the images.

Referring to FIG. 3, the method 200 may be performed by the ECU 25 of FIG. 1 as a series of steps or “logic blocks”, each of which is executable by the processor(s) 52 of the ECU 25. The method 200 according to the non-limiting exemplary embodiment of FIG. 3 commences with block B201 (“Illuminate Target Eye”) with illuminating the target eye 16. As shown in FIG. 1, the target eye 16 is illuminated with the illumination source 66 over a wideband spectrum of light, including light 65L and 67L. As this process occurs, the patient 18 should continue to focus on the optical target 61. The method 200 proceeds to block B202 as the respective visible and invisible light 65L and 67L falls incident upon the target eye 16.

At block B202 (“Capture Images”), the camera assembly 60 of FIG. 1 receives the visible reflected light 65R and invisible reflected light 67R. In response to receiving the reflected light 65R and 67R, the camera assembly 60 outputs corresponding visible spectrum images 71 and invisible spectrum images 73. The method 200 proceeds to block B204 once the ECU 25 has received data corresponding to the visible and invisible spectrum images 71 and 73 or has begun to receive a stream of such images according to a calibrated sampling frequency, such as 30, 60, or 120 fps.

Block B204 (“Generate Composite Image”) entails generating the at least one composite image 22 based on the visible light spectrum images 71 and the invisible spectrum images 73. For the example of the image sensor assembly 60 including the first perspective camera sensor 62-1 configured to capture first perspective images of the target eye 16 and the second perspective camera sensor 62-2 configured to capture second perspective images of the target eye 16, the first and second perspective camera sensors 62-1 and 62-2 are capable of capturing both visible light spectrum images 71 and invisible spectrum images 73. The ECU 25 is programmed to generate the at least one composite image 22 by organizing the first perspective images in both the visible and invisible light spectrum and the second perspective images in both the visible and invisible light spectrum each in an alternating sequential pattern.

Alternatively, if the image sensor assembly 60 is configured to capture a single perspective in the visible and invisible light spectrums, the ECU 25 is programmed to generate the at least one composite image 22 by organizing the visible spectrum images 71 in an alternating sequential pattern with the invisible spectrum images 73.

In yet another example, the ECU 25 is programmed to generate the at least one composite image 22 by organizing the visible spectrum images 71 adjacent to corresponding invisible spectrum images 73.

In a further example, the ECU 25 is programmed to generate the at least one composite image 22 by organizing corresponding image pairs from the visible light spectrum images 71 and the invisible spectrum images 73 in at least one of an overlaid image configuration or a blended image configuration.

The composite image 22 can be generated as discussed above depending on the type of images received and the desired output or surgical procedure being performed. Once the composite image 22 is generated, the method 200 proceeds to block B206.

Block B206 (“Display Composite Image”) entails displaying the composite image 22 generated from block B204. As discussed above, the composite image 22 can be displayed on the display 24 or the digital goggles 78 through the electronic display control signals CC₂₄.

Furthermore, the embodiments shown in the drawings, or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A visualization system for use during an ophthalmic procedure on a target eye comprising:
at least one illumination source configured to emit light in a visible light spectrum and in an invisible light spectrum;
a light filter located along a reflected light path and configured to allow for the transmission of light within a filtered light spectrum;
an image sensor assembly positioned along the reflected light path and configured to detect a first plurality of images from the visible light spectrum and a second plurality of images from the invisible light spectrum; and
an electronic control unit (ECU) in communication with the at least one illumination source and the image sensor assembly, wherein the ECU is programmed to:
activate the at least one illumination source to emit light in the visible light spectrum and in the invisible light spectrum;
receive image data from the image sensor assembly representative of the first plurality of images and the second plurality of images;
generate at least one composite image based on the first plurality of images and the second plurality of images; and
display the at least one composite image.

2. The visualization system of claim 1, wherein the invisible light spectrum includes an infrared light spectrum.

3. The visualization system of claim 1, wherein the invisible light spectrum includes an ultraviolet light spectrum.

4. The visualization system of claim 1, wherein the image sensor assembly includes a first sensor configured to capture a first perspective of the target eye and a second sensor configured to capture a second perspective of the target eye and the first plurality of images includes a first plurality of first perspective images and a first plurality of second perspective images and the second plurality of images includes a second plurality of first perspective images and a second plurality of second perspective images.

5. The visualization system of claim 4, wherein the ECU is programmed to generate the at least one composite image by organizing the first plurality of first perspective images and the second plurality of first perspective images in an alternating sequential pattern and organizing the first plurality of second perspective images and the second plurality of second perspective images in an alternating sequential pattern.

6. The visualization system of claim 1, wherein the ECU is programmed to generate the at least one composite image by organizing the first plurality of images in an alternating sequential pattern with the second plurality of images.

7. The visualization system of claim 1, wherein the ECU is programmed to generate the at least one composite image by organizing corresponding images from the first plurality of images adjacent corresponding images from the second plurality of images.

8. The visualization system of claim 1, wherein the ECU is programmed to generate the at least one composite image by organizing corresponding image pairs from the first plurality of images and the second plurality of images in an overlaid image configuration.

9. The visualization system of claim 1, wherein the ECU is programmed to generate the at least one composite image by organizing corresponding image pairs from the first plurality of images and the second plurality of images into a blended image configuration.

10. The visualization system of claim 1, wherein the at least one illumination source includes a first light source operable for directing visible light toward the target eye, the first light source including an array of red, green, and blue (RGB) laser diodes and a second light source operable for directing invisible light toward the target eye, the second light source including at least one an IR laser diode or a UV diode.

11. A non-transitory computer-readable storage medium embodying programmed instructions which, when executed by a processor, are operable for performing a method comprising:
illuminating a target eye by directing a visible spectrum light and an invisible spectrum light toward the target eye with at least one illumination source;
capturing a first plurality of images from the visible light spectrum and a second plurality of images from the invisible light spectrum with an image sensor assembly;
generating at least one composite image based on the first plurality of images and the second plurality of images; and
displaying the at least one composite image.

12. The computer-readable storage medium of claim 11, wherein
the image sensor assembly includes a first sensor configured to capture a first perspective of the target eye and a second sensor configured to capture a second perspective of the target eye and the first plurality of images includes a first plurality of first perspective images and a first plurality of second perspective images and the second plurality of images includes a second plurality of first perspective images and a second plurality of second perspective images; and
generating the at least one composite image includes organizing the first plurality of first perspective images and the second plurality of first perspective images in an alternating sequential pattern and organizing the first plurality of second perspective images and the second plurality of second perspective images in an alternating sequential pattern.

13. The computer-readable storage medium of claim 11, wherein generating the at least one composite image includes organizing the first plurality of images in an alternating sequential pattern with the second plurality of images.

14. The computer-readable storage medium of claim 11, wherein generating the at least one composite image includes organizing corresponding images from the first plurality of images adjacent corresponding images from the second plurality of images on a display.

15. The computer-readable storage medium of claim 11, wherein generating the at least one composite image includes organizing corresponding image pairs from the first plurality of images and the second plurality of images in one of an overlaid image configuration or a blended image configuration.

16. A method for using a visualization system during an ophthalmic procedure, the method comprising:
illuminating a target eye by directing a visible spectrum light and an invisible spectrum light toward the target eye with at least one illumination source;
capturing a first plurality of images from the visible light spectrum and a second plurality of images from the invisible light spectrum with an image sensor assembly;
generating at least one composite image based on the first plurality of images and the second plurality of images; and
displaying the at least one composite image.

17. The method of claim 16, wherein
the image sensor assembly includes a first sensor configured to capture a first perspective of the target eye and a second sensor configured to capture a second perspective of the target eye and the first plurality of images includes a first plurality of first perspective images and a first plurality of second perspective images and the second plurality of images includes a second plurality of first perspective images and a second plurality of second perspective images; and
generating the at least one composite image includes organizing the first plurality of first perspective images and the second plurality of first perspective images in an alternating sequential pattern and organizing the first plurality of second perspective images and the second plurality of second perspective images in an alternating sequential pattern.

18. The method of claim 16, wherein generating the at least one composite image includes organizing the first plurality of images in an alternating sequential pattern with the second plurality of images.

19. The method of claim 16, wherein generating the at least one composite image includes organizing corresponding images from the first plurality of images adjacent corresponding images from the second plurality of images on a display.

20. The method of claim 16, wherein generating the at least one composite image includes organizing corresponding image pairs from the first plurality of images and the second plurality of images in one of an overlaid image configuration or a blended image configuration.
